(19) United States
(12) Patent Application Publication
(10) Pub. No.: US 2005/0180991 A1
(43) Pub. Date: Aug. 18, 2005

(54) Hypotensive agent and food

(76) Inventor: Kenichi Matsunaga, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 10/915,420

(22) Filed: Aug. 11, 2004

(30) Foreign Application Priority Data
Feb. 12, 2004 (JP) 2004-035827

(51) Int. Cl. A61K 35/84

(52) U.S. Cl. 424/195.15

(57) ABSTRACT

A hypotensive agent and food are disclosed, which contain Tricholoma matsutake, in particular Tricholoma matsutake of the FERM BP-7304 strain, and any of mycelia, broths, or fruit bodies (including spores) thereof, as they are, dried products thereof, or extracts thereof (e.g., a hot water extract or an alkaline solution extract). Methods of treating hypertension by the use of the hypotensive agent and food are also disclosed.

### BACKGROUND OF THE INVENTION

1. Field of the Invention

The present invention relates to a hypotensive agent and food for the treatment of hypertension in animals and humans. The hypotensive agent and food of the present invention may be administered not only as a medicament but also in various forms, for example, as eatable and drinkable products such as health-promoting foods (specified health food and nutritional-functional food), as so-called health food (both including drinkable products), or as feeds. Further, the agent of the present invention may be administered in the form of an agent that is temporarily kept in the mouth but then spat out without the retention of most components, for example, a dentifrice, a mouthwash agent, a chewing gum, or a collutorium, or in the form of an inhalant drawn in through the nose.

2. Description of Related Art

Hypertension is regarded as one of the three major risk factors for heart diseases such as angina pectoris, cardiac infarction and cardiac failure. In addition, the disease is likely to cause kidney disease, resulting, in combination with other factors such as obesity, diabetes and hyperlipemia, in aggravation of hypertension. In fact, the number of hypertensives has been increasing every year; the Ministry of Health, Labor and Welfare estimate of 1998 shows that the number of hypertensive patients was approximately 7.7 million and the number of deaths due to hypertension was 6,700.

To inhibit development and progress of organ disorders due to hypertension through the maintenance of the normal blood pressure range, what is generally known as “hypotensive drugs (Ca antagonists, ACE inhibitors, AII receptor antagonists, diuretics, β blockers, α blockers, etc.)” have been used independently or in combination. However, there is a need to develop safe and new types of therapeutic and preventive agents and functional foods, because these hypotensive drugs have both merits and demerits.

Mushrooms have contributed to the health of Japanese people throughout history, due to their various physiological activities. For example, with respect to matsutake [Tricholoma matsutake (S. Ito & Imai) Sing.], JP-B-57-1230 (Kokoku) discloses that emitanine-5-A, emitanine-5-B, emitanine-5-C, and emitanine-5-D, which are separated and purified from a liquid extract obtained by extracting a liquid culture of Tricholoma matsutake mycelia with hot water or a diluted alkaline solution, exhibit activity of inhibiting the proliferation of sarcoma 180 cells. Further, JP Patent No. 2767521 discloses that a protein with a molecular weight of 0.2 to 0.21 million (a molecular weight of a subunit=0.1 to 0.11 million) that is separated and purified from an extract of Tricholoma matsutake fruit bodies with water exhibits antitumor activity.

Furthermore, the present inventors have found that a hot water extract of Tricholoma matsutake, an alkali-solution extract of Tricholoma matsutake, or an adsorption fraction of these extracts by an anion exchange resin has immuno-enhancing activity (PCT WO 01/49308 A1). The present inventors have also found that a partial purified fraction derived from particular mycelia of Tricholoma matsutake has activity of promoting recovery from stress loading (PCT WO 03/070264 A1).

### SUMMARY OF THE INVENTION

As described above, Tricholoma matsutake has been found to have various physiological activities, such as antitumor activity, immuno-enhancing activity, and activity of promoting recovery from stress loading. However, as far as the inventors know, there has been no report on the superior therapeutic effectiveness for hypertension of T. matsutake or other basidiomycetes of genus Tricholoma to which T. matsutake belongs.

After intensive studies with spontaneously hypertensive rats (SHRs), the inventors have found that T. matsutake and other basidiomycetes of genus Tricholoma exhibit an effect to inhibit the blood pressure increase in SHRs, and completed this invention.

Hence, an object of the invention is to provide hypotensive agents and foods utilizing basidiomycetes belonging to the genus Tricholoma, such as T. matsutake.

The present invention relates to a hypotensive agent containing Tricholoma matsutake or an extract thereof.

Further, the present invention relates to a hypotensive food containing Tricholoma matsutake or an extract thereof.

Further, the present invention relates to a method of the treatment of hypertension which comprises administrating to a human or an animal in an effective amount of the hypotensive agent.

Still further, the present invention relates to a method of the treatment of hypertension which comprises the intake of by a human or an animal in an effective amount of the hypotensive food.

The invention provides safe and stable- and massive-supply drugs and foods for the treatment of hypertension and the prevention of potential complications, such as kidney disease, and other diseases.

### BRIEF DESCRIPTION OF THE DRAWINGS

FIG. 1 is a graph showing body weight changes in each group during experiment in an example; and

FIG. 2 is a graph showing blood pressure and pulse rate changes in each group during experiment in an example.

### DETAILED DESCRIPTION OF THE INVENTION

Tricholoma matsutake [(S. Ito & Imai) Sing.] to be used for a hypotensive agent and food of the present invention can be used in any form of mycelia, broths, or fruit bodies and they can be used in either a fresh or dried state. In the present invention, fruit bodies include spores. Further, extracts from these mycelia, broths, and fruit bodies, may be used for the present invention.

In the present invention, the T. matsutake FERM BP-7304 strain is particularly preferably used.

The T. matsutake FERM BP-7304 strain was previously filed by the present applicant as a novel strain (PCT WO 02/30440 A1), and was deposited on Sep. 14, 2000, at Independent Administrative Institution, National Institute of Advanced Industrial Science and Technology (former National Institute of Bioscience and Human-Technology, Agency of Industrial Science and Technology, Japan). This T. matsutake FERM BP-7304 strain was a mycelium passage strain obtained by cutting out a fruit body tissue from the T. matsutake CM 6271 strain harvested in Kameoka, Kyoto, Japan, and culturing the tissue in a test tube. The FERM BP-7304 strain has been maintained in Biomedical Research Laboratories, Kureha Chemical Industries Co., Ltd.

The fruit body of the T. matsutake FERM BP-7304 strain had a fruit body form identical to a T. matsutake fruit body described on plate pages 9 and 26 of “Genshoku-nihon shin-kinrui zukan (1)” (edited by Rokuya Imaseki and Tsuguo Hongo, published by Hoikusha in 1957).

The T. matsutake FERM BP-7304 strain can be subcultured in a slant Ebios agar medium. After mycelia of the T. matsutake FERM BP-7304 strain is inoculated in a plate Ebios agar medium, white mycelia densely grow in a radial pattern, forming a large colony. When the colony is observed with a scanning electron microscope, an uncountable number of branched mycelia with a thickness of 1 to 2 μm are present and sometimes projections with a size of several μm are present on the side of the mycelia. For mass cultivation of the mycelia of the strain, the mycelia are inoculated on a liquid medium and cultured by stationary cultivation, shaking cultivation, tank cultivation, or the like.

It should be noted that the T. matsutake FERM BP-7304 strain can be maintained by subculture or cultured mostly in the form of mycelia, but it may also exist in the form of fruit body.

The mycological characteristics of the T. matsutake FERM BP-7304 strain are described below.

(1) Cultural and morphological characteristics in malt extract agar medium:

White hyphae grew densely and radially, forming a colony. The diameter of the colony on the 30th day after inoculation was about 4 cm.

(2) Cultural and morphological characteristics in Czapeck agar medium, oatmeal agar medium, synthetic mucor agar medium, and phenoloxidase reaction assay medium:

Almost no growth of hyphae was observed in any of the above media even after 1 month had passed since inoculation.

(3) Cultural and morphological characteristics in YpSs agar medium:

The T. matsutake FERM BP-7304 strain grew in a mat shape having a white gloss. On the 30th day after inoculation, the growth distance was about 5 mm.

(4) Cultural and morphological characteristics in glucose dry yeast agar medium:

The T. matsutake FERM BP-7304 strain grew in a mat shape having a white gloss. On the 30th day after inoculation, the growth distance was about 2 mm.

(5) Optimum growth temperature and growth range:

In a 100-mL Erlenmeyer flask containing 10 mL of sterilized liquid medium (3% glucose, 0.3% yeast extract, pH 7.0), about 2 mg of seed fungi of the T. matsutake FERM BP-7304 strain was each inoculated and cultured at various temperatures of 5 to 35° C. On 28th day of incubation, fungus bodies were taken out from the flask, washed well with distilled water, and then dried for mass measurement. The results show that the mass of the fungus bodies linearly increased within the temperature range of 5 to 15° C. and leniently increased within the temperature range of 15 to 25° C. Almost no fungi grew at temperatures of 27.5° C. or more. The optimum temperature for growth is from 15 to 25° C.

(6) Optimum growth pH and growth range:

Liquid media (3% glucose, 0.3% yeast extract) were adjusted with 1 mol/L hydrochloric acid or 1 mol/L potassium hydroxide so that the media having various pH levels from 3.0 to 8.0 were prepared to determine the pH for fungus body growth. Namely, each medium was sterilized with a filter, and 10 mL of the sterilized medium was dispensed into a 100-mL sterilized Erlenmeyer flask. About 2 mg of seed fungi of the T. matsutake FERM BP-7304 strain was inoculated in the flask and cultured at 22° C. Thereafter, fungus bodies were taken out from the flask, washed well with distilled water, and then dried for mass measurement. The results show that the pH growth limit for the fungus bodies was from 3.0 to 7.0 and the optimum pH for growth was 4.0 to 6.0.

(7) Formation of zone line by dual culture:

On an Ebios plate agar medium, a block (about 3 mm×3 mm×3 mm) of the T. matsutake FERM BP-7304 strain and each block (about 3 mm×3 mm×3 mm) of 13 kinds of known T. matsutake strains (for example, IFO 6915 strain; Institute for Fermentation Osaka) were placed with about 2 cm of distance between each strain, and cultured at 22° C. for 3 weeks. Thereafter, it was determined whether a zone line was formed on the boundary between two colonies among them.

The results show that the T. matsutake FERM BP-7304 strain did not form definite zone lines against all of the known T. matsutake strains (13 kinds). It is considered that no zone line is formed by dual culture between different strains of T. matsutake, and among the known T. matsutake strains (13 kinds) there was no combination of strains that formed a definite zone line therebetween. Therefore, it is considered the strains are compatible one another.

(8) Nutritional requirement:

About 2 mg of seed fungi of the T. matsutake FERM BP-7304 strain was inoculated in a 100-mL Erlenmeyer flask containing 10 mL of sterilized synthetic medium for mycorrhizal fungus (Ohta medium, Ohta et al. “Trans. Mycol. Soc. Jpn.,” 31, 323-334, 1990), and cultured at 22° C. On 42nd day of culturing, fungus bodies were taken out from the flask, washed well with distilled water, and dried for mass measurement. Consequently, 441 mg of fungus body was obtained.

Instead of glucose in the above synthetic medium for mycorrhizal fungus as a carbon (C) source, any one of 28 kinds of carbohydrate-related substances was added to each medium. The T. matsutake FERM BP-7304 strain was inoculated and cultured on each medium, and after the completion of culture the mass of fungus bodies was measured. As a result, the carbohydrate-related substances are listed below in descending order corresponding to the fungus body mass:

Wheat starch>corn starch>dextrin>methyl β glucoside>cellobiose>mannose>fructose>arabinose>sorbitol>glucose>lactose>glycogen>mannitol>ribose>maltose>trehalose>galactose>raffinose>melibiose>N-acetylglucosamine.

Incidentally, almost no growth of the fungi was observed in cellulose, dulcitol, sucrose, xylose, methyl a glucoside, inulin, inositol, or sorbose.

Next, instead of ammonium tartrate in the above synthetic medium for mycorrhizal fungus as a nitrogen (N) source, any one of 17 kinds of nitrogen-related substances was added to each medium. The T. matsutake FERM BP-7304 strain was inoculated and cultured on each medium, and after the completion of culture the mass of fungus bodies was measured. As a result, the nitrogen-related substances are listed below in descending order corresponding to the fungus body mass:

Corn steep liquor>soy peptone>milk peptone>ammonium nitrate>ammonium sulfate>ammonium tartrate>ammonium carbonate>asparagine>ammonium phosphate>ammonium chloride>sodium nitrate>meat extract>yeast extract>casamino acid>chlorella>triptone>potassium nitrate.

Further, among minerals and vitamins in the above synthetic medium, a medium was prepared without a particular single component. The T. matsutake FERM BP-7304 strain was inoculated and cultured on that medium, and after the completion of culture the mass of fungus bodies was measured.

As a result, even when any one of calcium chloride.dihydrate, manganese (II) sulfate.pentahydrate, zinc sulfate.heptahydrate, cobalt sulfate.heptahydrate, copper sulfate.pentahydrate, nickel sulfate.hexahydrate, amine hydrochloride, nicotinic acid, folic acid, biotin, pyridoxine hydrochloride, carnitine chloride, adenine sulfate.dihydrate, and choline hydrochloride was removed from the medium, the fungus body mass was almost uneffected.

On the other hand, when any one of magnesium sulfate.heptahydrate, iron (II) chloride, and potassium dihydrogen phosphate was removed, the fungus body mass remarkably reduced. In other words, magnesium, iron, phosphorus, and potassium are considered essential for the growth of the T. matsutake FERM BP-7304 strain.

(9) DNA base composition (GC content):

The GC content was 49.9%.

(10) DNA pattern prepared by RAPD method:

In terms of DNA patterns prepared by the RAPD (Random Amplified Polymorphic DNA) method independently using 6 different kinds of PCR (Polymerase Chain Reaction) primers (10 mer), the T. matsutake FERM BP-7304 strain was compared with 44 kinds of known T. matsutake strains (for example, the IFO 6915 strain; Institute for Fermentation Osaka). The T. matsutake FERM BP-7304 strain exhibited a DNA pattern different from all of the other known T. matsutake strains (44 kinds).

Preferable embodiments of the hypotensive agent and food of the present invention contain as an active ingredient: (i) fresh mushrooms of T. matsutake FERM BP-7304 strain (e.g., mycelia, broths, or fruit bodies of the strain) or a dried powder thereof; (ii) a hot water extract of the T. matsutake FERM BP-7304 strain (e.g., a hot water extract of mycelia, broths, or fruit bodies of the strain); or (iii) an alkaline solution extract of the T. matsutake FERM BP-7304 strain (e.g., an alkaline solution extract of mycelia, broths, or fruit bodies of the strain). However, the active ingredient is not limited to these embodiments.

For the present invention, the above embodiment (i) is preferable.

As mycelia of the T. matsutake FERM BP-7304 strain usable as the active ingredient of the hypotensive agent and food of the present invention, mycelia may be used, for example, in a form obtained directly by removing a medium from a mixture of mycelia obtained by culturing (that is, cultured mycelia) and a medium with an appropriate removing means (e.g., filtration). Alternatively, dried mycelia, which are obtained by removing water from the mycelia after the removal of the medium with an appropriate removing means (e.g., lyophilization) may be used. Further, dried mycelia powders, which are obtained by grinding the above dried mycelia, may be used.

As broths of the T. matsutake FERM BP-7304 strain usable as the active ingredient of the hypotensive agent and food of the present invention, a broth may be used, for example, in the form of a mixture of mycelia obtained by cultivation (that is, cultured mycelia) and a medium. Alternatively, a dried broth obtained by removing water from the above mixture with an appropriate removing means (e.g., lyophilization) may be used. Further, dried broth powders, which are obtained by grinding the above dried broth, may be used.

A method for the above-described cultivation is not particularly limited, and any of the ordinary methods for culturing T. matsutake fungi can be used. However, a method, for example, disclosed in JP Patent Application No. 2002-311840 is preferably employed, since the method enables mass production without the loss of the physiological activities of matsutake fungi. The method comprises: a step for obtaining matsutake fungi II by culturing or preserving the T. matsutake FERM BP-7304 strain (“matsutake fungi I”) in a solid or liquid medium; a step for obtaining matsutake fungi III by stationary liquid-cultivation of the matsutake fungi II; a step for obtaining matsutake fungi IV by shaking cultivation of the matsutake fungi III; a step for obtaining matsutake fungi V by stirring-culture of the matsutake fungi IV with the use of a small culture apparatus with a volume of less than 100 L without the aeration in a liquid medium; a step for obtaining matsutake fungi VI by deep stirring-culture of the matsutake fungi V with the use of a medium- or large-sized culture apparatus with a volume of 100 L or more; a step for obtaining matsutake fungi VII by deep stirring-culture of the matsutake VI with the use of a medium- or large-sized culture apparatus with a volume of 100 L or more; and a step for obtaining matsutake fungi VIII by deep stirring-culture of the matsutake fungi VII with the use of a medium- or large-sized culture apparatus with a volume of 100 L or more.

<Step for Obtaining matsutake Fungi II by Culturing or Preserving matsutake Fungi I>

A medium to be used herein is not particularly limited, as long as such medium is a common one containing a nutrient substrate for culturing matsutake fungi. Examples thereof include an Ohta medium (Ohta et al., “Trans. Mycol. Soc. Jpn.,” 31, 323-334, 1990), an MMN medium (Marx, D. H., “Phytopathology,” 59: 153-163, 1969), and a Hamada medium (Hamada, “Matsutake, 97-100, 1964), but the usable medium is not limited to these examples.

Preferable examples of a solidifying agent for a solid medium include carrageenan, mannnan, pectin, agar, curdlan, starch, and alginic acid. Among these, agar is preferable.

Examples of usable nutrient substrate for a medium include a carbon source, a nitrogen source, and an inorganic element source.

Examples of the above carbon source include: starches, such as rice starch, wheat flour starch, potato starch, and sweet potato starch; polysaccharides, such as dextrin and amylopectin; oligosaccharides, such as maltose and sucrose; and monosaccharides, such as fructose and glucose. Examples thereof further include malt extracts. Depending on the growth speed of matsutake fungi, matsutake has a period in which monosaccharides such as glucose are preferably used and a period in which starches are preferably used. Therefore, a suitable carbon source is selected based on the period, and if necessary, these carbon sources may be used in combination.

Examples of the above nitrogen source include naturally occurring substances such as yeast extracts, dried yeast, corn steep liquor, soy flour, and soy peptone, ammonium nitrate, ammonium sulfate, and urea. These may be used either alone or in combination. In general, considering growth speed, naturally occurring substances, particularly yeast extracts, are preferable.

The inorganic element source is used to supply phosphoric acid and trace elements. Examples thereof include, in addition to phosphates, inorganic salts (e.g., sulfates, hydrochlorides, nitrates, and phosphates) of metal ions such as sodium, potassium, magnesium, calcium, zinc, manganese, copper, andiron. A required amount of the inorganic element is dissolved in a medium.

In addition, vitamins such as vitamin B₁ or amino acids may be added to the medium.

Further, in accordance with the properties of matsutake fungi to be used, plant extracts, organic acids, nucleic acid-related substances or the like may be added. Examples of the plant extracts include extracts of fruit crops, root crops, and leaf vegetables. Examples of the organic acids include citric acid, tartaric acid, malic acid, fumaric acid, and lactic acid. Examples of the nucleic acid-related substances include commercially available nucleic acids, nucleic acid extracts, yeast, and yeast extracts.

In preparing a solid medium, the amount of carbon source to be used is preferably 10 to 100 g/L, more preferably 10 to 50 g/L, and most preferably 20 to 30 g/L.

The amount of nitrogen source to be used is in nitrogen equivalent, preferably 0.005 to 0.1 mol/L, more preferably 0.007 to 0.07 mol/L, and most preferably 0.01 to 0.05 mol/L.

The amount of phosphate to be used is in phosphorus equivalent, preferably 0.001 to 0.05 mol/L, more preferably 0.005 to 0.03 mol/L, and most preferably 0.01 to 0.02 mol/L. In addition, other inorganic salts, vitamins, plant extracts, organic acids, nucleic acid-related substances, or the like may be optionally added in accordance with the properties of the matsutake fungi. Furthermore, the prepared nutrient substrate solution is adjusted so as to have a pH of preferably 4 to 7, more preferably 4.5 to 6.0, and most preferably 5.0 to 5.5.

<Stationary Liquid Cultivation>

Next, a method for producing matsutake fungi III by stationary cultivation of matsutake fungi II (which was obtained by culturing or preserving of matsutake fungi I in a solid or liquid medium) in a liquid medium will be described.

Usually, an Erlenmeyer flask with a volume of 100 mL to 2 L is used.

The stationary liquid cultivation starts by inoculating matsutake fungi II on the liquid medium.

The liquid medium is used, in which the ratio (“magnification at the time of inoculation”) of a mixture of the culture liquid containing the matsutake fungi II with a liquid medium to the culture liquid containing the matsutake fungi II is preferably 2:1 to 50:1, and more preferably 3:1 to 30:1.

The culture liquid containing the matsutake fungi II is inoculated on the liquid medium so that the ratio (“concentration of initial mycelia”) between the mass of dried mycelia of matsutake fungi II in the culture liquid containing the matsutake fungi II and the volume of the mixture of the culture liquid containing the matsutake fungi II with the liquid medium becomes preferably 0.05 to 3 g/L, and more preferably 0.1 to 2 g/L.

The temperature for the stationary liquid cultivation is preferably 15 to 30° C., and more preferably 20 to 25° C., and the cultivation period is preferably 30 to 400 days and more preferably 120 to 240 days. If the cultivation period is less than 30 days or more than 400 days, it is difficult to obtain matsutake fungi III having growth ability suitable for mass culture.

In terms of growth ability, the culturing is preferably performed so that the dried mycelia content (unit: g/L) in the culture liquid after the stationary liquid cultivation becomes 2 to 25 times (in a ratio referred to as “mycelia increase ratio”) greater than the concentration of initial mycelia.

The liquid medium to be used for the stationary liquid cultivation contains a nutrient substrate so that the medium has an osmotic pressure of preferably 0.01 to 0.8 MPa, more preferably 0.02 to 0.7 MPa, and most preferably 0.03 to 0.5 MPa.

As the nutrient source to be used for the stationary liquid cultivation, the same carbon source, nitrogen source, inorganic element source, vitamins such as vitamin B₁, amino acids, and the like can be used as those used for the solid medium for culturing matsutake fungi I.

The amount of carbon source to be used is preferably 10 to 100 g/L, more preferably 20 to 60 g/L, and most preferably 25 to 45 g/L. Generally, monosaccharides such as glucose are used.

The amount of nitrogen source to be used is in nitrogen equivalent, preferably 0.005 to 0.1 mol/L, more preferably 0.007 to 0.07 mol/L, and most preferably 0.01 to 0.05 mol/L.

When phosphates are used, the amount thereof to be used is in phosphorus equivalent, preferably 0.001 to 0.05 mol/L, more preferably 0.005 to 0.03 mol/L, and most preferably 0.01 to 0.02 mol/L.

In addition, other inorganic salts, vitamins, plant extracts, organic acids, nucleic acid-related substances, or the like may be properly added in accordance with the properties of matsutake fungi.

The prepared nutrient substrate solution has a pH of preferably 4 to 7, more preferably 4.5 to 6.5, and most preferably 5.0 to 6.0.

A part or the whole of the culture liquid containing matsutake fungi III by stationary liquid cultivation may be used again as an inoculation source for stationary liquid cultivation in the stationary liquid cultivation step in the same manner as the culture liquid (or culture product) containing matsutake fungi II.

<Shaking Cultivation>

Next, a method for producing matsutake fungi IV by shaking cultivation of matsutake fungi III (which was obtained by stationary cultivation of matsutake fungi II) will be described.

In general, an Erlenmeyer flask with a volume of 300 mL to 5 L is used.

The shaking cultivation starts by inoculating matsutake fungi III on a liquid medium.

The liquid medium is used, in which the ratio (“magnification at the time of inoculation”) of a mixture of the culture liquid containing the matsutake fungi III with a liquid medium to the culture liquid containing the matsutake fungi III is preferably 2:1 to 50:1, and more preferably 3:1 to 30:1.

Further, in order to secure enough amount of the culture liquid to meet the magnification at the time of inoculation, the stationary liquid culture may be produced using a plurality of culture apparatuses.

The culture liquid containing the matsutake fungi III is inoculated on the liquid medium so that the ratio (concentration of initial mycelia”) between the mass of dried mycelia of matsutake fungi III in the culture liquid containing the inoculated matsutake fungi III and the volume of the mixture of the culture liquid containing the inoculated matsutake fungi III with the liquid medium becomes preferably 0.05 to 3 g/L, more preferably 0.1 to 2 g/L.

In the shaking cultivation, the temperature is preferably 15 to 30° C. and more preferably 20 to 25° C., and the culture period is preferably 7 to 50 days and more preferably 14 to 28 days.

As power required for the shaking culture, a power of 0.05 to 0.4 kW/m³ for shaking a unit volume of the culture liquid in the Erlenmeyer flask is generally used.

In terms of growth ability, the cultivation is preferably performed so that the dried mycelia content (unit: g/L) in the culture liquid after the stationary liquid cultivation becomes 2 to 25 times (in a ratio referred to as “mycelia increase ratio”) greater than the concentration of initial mycelia.

The liquid medium to be used for the shaking cultivation contains a nutrient substrate so that the medium has an osmotic pressure of preferably 0.01 to 0.8 MPa, more preferably 0.02 to 0.7 MPa, and most preferably 0.03 to 0.5 MPa.

As the nutrient source to be used for the shaking culture, the same carbon source, nitrogen source, inorganic element source, vitamins such as vitamin B₁, amino acids, and the like can be used as those used for the liquid medium for culturing matsutake fungi II.

The amount of carbon source to be used is preferably 10 to 100 g/L, more preferably 20 to 60 g/L, and most preferably 25 to 45 g/L. Generally, monosaccharides such as glucose are used.

The amount of nitrogen source to be used is in nitrogen equivalent, preferably 0.005 to 0.1 mol/L, more preferably 0.007 to 0.07 mol/L, and most preferably 0.01 to 0.05 mol/L.

The amount of phosphate salts to be used is in phosphorus equivalent, preferably 0.001 to 0.05 mol/L, more preferably 0.005 to 0.03 mol/L, and most preferably 0.01 to 0.02 mol/L.

In addition, other inorganic salts, vitamins, amino acids, plant extracts, organic acids, nucleic acid-related substances, or the like may be properly added in accordance with the properties of the matsutake fungi.

The prepared nutrient substrate solution has a pH of preferably 4 to 7, more preferably 4.5 to 6.5, and most preferably 5.0 to 6.0.

<Stirring Cultivation>

Next, a method for producing matsutake fungi V, matsutake fungi VI, matsutake fungi VII, and matsutake fungi VIII by stirring cultivation will be described.

The stirring cultivation starts by inoculating matsutake fungi (IV to VII) on a liquid medium. In the description below, T. matsutake IV refers to T. matsutake obtained from shaking culture of T. matsutake III; T. matsutake V refers to T. matsutake obtained from non-aerated spinner culture of T. matsutake IV using a small culturing apparatus of less than 100 L; T. matsutake VI refers to T. matsutake obtained from deep spinner culture of T. matsutake V using a medium to large culturing apparatus of 100 L or more; T. matsutake VII refers to T. matsutake obtained from deep spinner culture of T. matsutake VI using a medium to large culturing apparatus of 100 L or more; and T. matsutake VIII refers to T. matsutake obtained from deep spinner culture of T. matsutake VII using a medium to large culturing apparatus of 100 L or more.

The liquid medium to be used for the stirring cultivation is prepared in the following manner.

As a nutrient substrate, the same carbon source, nitrogen source, inorganic element source, vitamins such as vitamin B₁, and amino acids may be used as those used for the shaking cultivation.

The amount of carbon source to be used is preferably 10 to 100 g/L, more preferably 20 to 60 g/L, and most preferably 25 to 45 g/L. Starches are preferably used.

When monosaccharides such as glucose, which affects the osmotic pressure of the culture liquid to be stirred, are used in combination, the amount thereof to be used is preferably 0.1 to 60 g/L, more preferably 0.5 to 40 g/L, and most preferably 0.7 to 20 g/L.

The amount of nitrogen source to be used is in nitrogen equivalent, preferably 0.005 to 0.1 mol/L, more preferably 0.007 to 0.07 mol/L, and most preferably 0.01 to 0.05 mol/L.

The amount of phosphates to be used is in phosphorus equivalent, preferably 0.001 to 0.05 mol/L, more preferably 0.005 to 0.03 mol/L, and most preferably 0.01 to 0.02 mol/L.

Further, other inorganic salts, vitamins, amino acids, plant extracts, organic acids, nucleic acid-related substances, and the like may be properly added in accordance with the properties of matsutake fungi.

The pH of the prepared nutrient substrate solution is preferably 4 to 7, more preferably 4.5 to 6.5, and most preferably 5.0 to 6.0. The liquid medium to be used for stirring cultivation contains a nutrient substrate so that it has an osmotic pressure of preferably 0.01 to 0.8 MPa, more preferably 0.02 to 0.7 MPa, and most preferably 0.03 to 0.5 MPa.

The temperature for the stirring cultivation is 15 to 30° C., preferably 20 to 25° C.

The liquid medium is used, in which the ratio (“magnification at the time of inoculation”) of a mixture of the culture liquid containing the matsutake fungi (IV to VII) with the liquid medium to the culture liquid containing the inoculated matsutake fungi (IV to VII) is preferably 2:1 to 50:1, more preferably 3:1 to 30:1, and most preferably 5:1 to 10:1.

The culture liquid containing the matsutake fungi (IV to VII) is inoculated on the liquid medium so that the volume ratio (“concentration of initial mycelia”) between the mass of dried mycelia of matsutake fungi (IV to VII) in the culture liquid containing inoculated matsutake fungi (IV to VII) and the mixture of the culture liquid containing the inoculated matsutake fungi (IV to VII) with the liquid medium becomes preferably 0.01 to 5 g/L, more preferably 0.05 to 3 g/L, and most preferably 0.1 to 2 g/L.

When matsutake fungi (V to VII) obtained by the stirring culture is used as mother fungi for stirring cultivation, the cultivation period is preferably 3 to 20 days, and particularly preferably 5 to 14 days.

After the cultivation period, the culture liquid contains matsutake fungi (V to VII), which have growth ability suitable for stirring cultivation, at amounts equivalent to dried mycelia content of preferably 0.5 to 10 g/L, more preferably 1 to 8 g/L, and most preferably 1 to 6 g/L.

In terms of growth ability, the culture is preferably performed so that the dried mycelia content (unit: g/L) in the culture liquid after the stationary liquid cultivation becomes 2 to 25 times (in a ratio referred to as “mycelia increase ratio”) greater than the concentration of initial mycelia.

The cultivation period for isolating matsutake mycelia from the matsutake fungi (V to VIII) obtained by the stirring cultivation is 5 to 30 days, more preferably 7 to 20 days, and most preferably 10 to 15 days.

During the above cultivation periods, the time when the assimilation speed of the carbon source decreases remarkably is considered to be the preferable time for terminating the cultivation. However, the time for terminating the cultivation can be properly determined in accordance with production patterns such as production cycle and production cost.

In terms of industrial production, the cultivation is preferably performed so that the dried mycelia content (unit: g/L) in the culture liquid after the stationary liquid cultivation becomes 35 to 100 times (in a ratio referred to as “mycelia increase ratio”) greater than the concentration of initial mycelia.

The culture liquid containing matsutake fungi IV produced by stirring cultivation may be used for a stirring cultivation step with the use of a culture apparatus such as a medium- or large-sized culture tank with a volume of 100 L or more.

The culture apparatus to be used for stirring cultivation is not particularly limited as long as the apparatus is capable of aeration-cultivation and maintaining sterility. As occasion demands, an apparatus that enables aeration or that can be installed with an aeration apparatus may be used. Therefore, an ordinary small-, medium-, and large-sized culture tank, or a jar fermentor, can be used.

In producing matsutake fungi V by culturing matsutake IV by the use of a jar fermentor or a small-sized culture tank with a volume of less than 100 L, the stirring cultivation is performed preferably without aeration in the liquid medium. The reason is that when the cultivation is performed with aeration in a jar fermentor or small-sized culture tank with a volume of less than 100 L, mycelia grow closely to each other to lose their growing points and their growing ability of inoculated fungi is damaged.

Further, when the cultivation with deep stirring is performed at industrial scale by the use of a culture apparatus such as a medium- or large-sized culture tank with a volume of 100 L or more, aeration is carried out when needed. In this case, the aeration volume is 0.05 to 1.0 vvm, and in particular preferably 0.2 to 0.5 vvm.

The stirring in the stirring cultivation is controlled by a stirring power required for a unit volume of the culture liquid at an early stage of the cultivation. Generally, by stirring within a power range of preferably 0.01 to 2 kW/m³ and more preferably 0.05 to 1 kW/m³, matsutake mycelia grow favorably. After the early stage, the fungi start to grow, thereby causing insufficient oxygen supply. Further, grown mycelia do not disperse adequately, and thus a larger strength of stirring is properly required. For the deep stirring, preferably, early stage cultivation is conducted with low aeration at low stirring speed and late stage cultivation is performed with high aeration at high stirring speed.

The separation and harvest of matsutake mycelia obtained by the deep stirring cultivation may be carried out by conventional methods. Examples of these methods include filtration by a filter press or the like, and centrifugation.

The obtained mycelia are preferably washed well with, for example, distilled water, and then provided for the subsequent hot water extraction step. Further, in order to enhance the extraction efficiency, the mycelia are preferably processed into crushed materials or powders.

As the fruit bodies of the T. matsutake FERM BP-7304 strain usable as the active ingredient of the hypotensive agent and food of the present invention, for example, fruit bodies as they are, or crushed fruit bodies, can be used. Alternatively, dried fruit bodies obtained by removing water therefrom with an appropriate removing means (e.g., lyophilization), may be used. Further, dried fruit body powders obtained by grinding the above dried fruit bodies may be used.

The hot water extract of the T. matsutake FERM BP-7304 strain usable as the active ingredient of the hypotensive agent and food of the present invention can be prepared by, for example, extracting mycelia, (i.e., the cultured mycelia), broths, or fruit bodies obtained by culturing the T. matsutake FERM BP-7304 strain with hot water.

The temperature of hot water to be used for the hot water extraction is not particularly limited, as long as the component that is contained in the T. matsutake FERM BP-7304 strain and that exhibits hypotensive activity is sufficiently extracted so as to result in the hot water extract. However, the temperature is preferably about 60 to 100° C., and more preferably about 80 to 98° C.

When mycelia or fruit bodies are used for the hot water extraction, it is preferable to process them into crushed materials or powders to enhance the extraction efficiency.

Further, it is preferable to carry out the hot water extraction step while stirring or shaking to improve the extraction efficiency. The period for extraction may be properly determined in accordance with, for example, the form of mycelia (e.g., a processed state when they are processed into a crushed or pulverized form), the temperature of the hot water, or treatment conditions with or without stirring or shaking. However, it is usually about 1 to 6 hours, and preferably about 2 to 3 hours.

The obtained hot water extract may be used as it is, namely, in a state containing insolubles, as the active ingredient of the hypotensive agent of the present invention. Alternatively, it may be used as the active ingredient of the hypotensive agent of the present invention after the insolubles and then low molecular weight fractions (preferably fractions containing substances with a molecular weight of 3500 or less) are removed from the extract.

The alkaline solution extract of the T. matsutake FERM BP-7304 strain usable as the active ingredient of the hypotensive agent and food of the present invention may be prepared by, for example, a method similar to the above-mentioned method for preparing the hot water extract of T. matsutake FERM BP-7304 strain, except that an alkaline solution is used instead of hot water.

An alkaline solution to be used for the alkaline solution extraction is not particularly limited, but, for example, hydroxides of alkaline metals (sodium, potassium, etc.), and in particular an aqueous solution of sodium hydroxide, may be used. The alkaline solution preferably has a pH of 8 to 13, and more preferably 9 to 12. The alkaline solution extraction is conducted preferably at a temperature of about 0 to 30° C., more preferably about 0 to 25° C. A period for extraction may be properly determined in accordance with, for example, the state of the mycelia residue (e.g., a processed state when the mycelia are processed into a crushed or pulverized form), the pH value or the temperature of the alkaline solution, or treatment conditions with or without stirring or shaking, but it is usually about 30 minutes to 5 hours, and preferably about 1 to 3 hours. The obtained alkaline solution extract may be directly used, or, if desired, subjected to neutralization treatment, and then used for the hypotensive agent and food of the present invention.

The hypotensive agent and food of the present invention can be administered to animals or humans, having as the active ingredient T. matsutake, in particular the T. matsutake FERM BP-7304 strain, or an extract thereof, either alone or, if desired, in combination with a pharmaceutically acceptable carrier.

In the invention, the term “hypotensive” or “antihypertensive” refers to the treatment of hypertensive symptoms (disease condition) in animals and humans, including retardation and inhibition of progress of hypertensive symptoms, as well as the prevention of potential complications induced by hypertension. Therefore, the dosage and administration of hypotensive drugs and foods of the invention is not specially limited, and it is preferable that these products be taken on a daily and continuous basis.

The formulation for administration and intake of the hypotensive agent and food of the present invention is not particularly limited to, but may be, for example, oral medicines such as powders, fine particles, granules, tablets, capsules, suspensions, emulsions, syrups, extracts or pills, or parenteral medicines such as injections, liquids for external use, ointments, suppositories, creams for topical application, or eye lotions.

The oral medicines may be prepared by conventional methods using, for example, fillers, binders, disintegrating agents, surfactants, lubricants, flowability-enhancers, diluting agents, preservatives, coloring agents, perfumes, tasting agents, stabilizers, humectants, antiseptics, and antioxidants. Examples of the aforementioned include gelatin, sodium alginate, starch, corn starch, saccharose, lactose, glucose, mannitol, carboxylmethylcellulose, dextrin, polyvinyl pyrrolidone, crystalline cellulose, soybean lecithin, sucrose, fatty acid esters, talc, magnesium stearate, polyethylene glycol, magnesium silicate, silicic anhydride, and synthetic aluminum silicate.

The parenteral administration may take the form of, for example, an injection such as a subcutaneous or intravenous injection, or rectal administration. Among the parenteral formulations, an injection is preferably used.

In preparing injections, for example, water-soluble solvents, such as physiological saline or Ringer's solution, water-insoluble solvents, such as plant oil or fatty acid esters, isotonizing agents such as glucose or sodium chloride, solubilizing agents, stabilizing agents, antiseptics, suspending agents, or emulsifying agents may be optionally used, in addition to the active ingredient.

The hypotensive agent and food of the present invention may be administered in the form of a sustained release preparation using sustained release polymers. For example, the hypotensive agent and food of the present invention may be incorporated in a pellet made of ethylenevinyl acetate polymers, and the pellet may be surgically implanted in a tissue to be treated or which is to be protected from cancer.

The hypotensive agent and food of the present invention contain as the active ingredient T. matsutake FERM BP-7304 strain or extracts thereof, or the like in amounts of 0.01 to 99% by mass, and preferably 0.1 to 90% by mass. However, amounts are by no means limited to the aforementioned.

A dose for administration or intake of the hypotensive agent and food of the present invention may be properly determined depending on the kind of disease, the age, sex, body weight, symptoms of a patient, method of administration or intake. The hypotensive agent and food of the present invention may be orally or parenterally administered or taken.

The form of administration or intake is not limited to a medicament, but various forms are available, such as eatable or drinkable products such as health-promoting foods (specified health foods and nutritional-functional foods), as so-called health foods (both including drinkable products), or as feeds. Further, the hypotensive agent and food of the present invention may be administered in the form of an agent that is temporarily kept in the mouth, but then spat out without the retention of most components, for example, a dentifrice, a mouthwash agent, a chewing gum, or a collutorium, or in the form of an inhalant drawn in through the nose. For example, the active ingredient such as T. matsutake FERM BP-7304 strain or extracts thereof may be added to a desired food (including a drink), a feed, a dentifrice, a mouthwash agent, a chewing gum, a collutorium, or the like as an additive (such as a food additive).

In the above description, the term “specified health food” means a food, for which it is permitted to indicate health functions possessed by that food (permission by Ministry of Health, Labor, and Welfare is required for each food). The term “nutritional-functional food” means a food, for which it is allowed to explicitly state the functions of nutritional components (the standard prescribed by Ministry of Health, Labor, and Welfare should be satisfied). The term “health food” widely means foods in general other than the above-mentioned health-promoting foods, and health food includes health supplements.

### EXAMPLES

The present invention will be described in detail by referring to the following Examples, but the technical scope of the present invention is not limited by these Examples.

I. Study Material

(1) Preparation of the Dried Mycelial Powder (Hereafter Referred to as “CM6271”) of T. matsutake FERM BP-7304

The mycelia of T. matsutake FERM BP-7304 was inoculated into 3.5 tons of sterilized medium (3% glucose, 0.3% yeast extract, pH 6.0) in a 7-ton culture tank and incubated at 25° C. for 4 weeks with agitation. The culture thus obtained was filtered to separate the mycelia, which was then rinsed thoroughly with distilled water.

A portion (approx. 1 kg) of the mycelia was frozen at −60° C., then freeze-dried with a lyophilizer (MINIFAST MOD. DO. 5; Edwards Corp.) to give a dried mycelia of 110 g.

The dried mycelia was pulverized with a homoblender (Wonder Blender Corp.) to give 100 g of dried powder (CM6271). The dried powder was stored in a container containing silica gel at 18° C.

(2) Reagents

The hypotensive drug used was hydralazine, which was purchased from Sigma Chemicals Co., US.

II. Administered Samples

A given quantity of CM6271 was weighed, diluted with water for injection JP to 6 w/v %, and homogenized for approximately 10 seconds for adequate dispersion.

Hydralazine was added to and suspended in the vehicle (solvent) containing 0.5 w/v % CMC-Na (carboxymethylcellulose sodium) to make a 0.5 w/v % solution.

The 6 w/v % solution of CM6271 was prepared everyday before administering to rats. The 0.5 w/v % solution of hydralazine was prepared once a week.

III. Animals and the Raising Environment

Forty-eight 8-week-old male SHR (spontaneously hypertensive rat) SPF (specific pathogen free) rats, purchased from Japan SLC, Inc., were used in the study.

During a 16 to 17-day acclimatization period for handling, clinical signs were observed everyday, and the rats were weighed on the following day of arrival and the last day of acclimatization.

During the late acclimatization period, blood pressure and pulse rate were measured once. Administration started when rats were 10-week old and weighed 197 to 255 g.

All the rats were individually raised in metal cages (W15×D30×H17 cm) placed in a barrier system feeding chamber set at 22±1° C. temperature, 55±10% relative humidity, 10 to 20 times/hour air change, and 6 to 18 hr lighting conditions. The temperature and relative humidity during the feeding period were maintained at 21.5 to 22.5° C. and 48 to 71%, respectively.

The rats were allowed ad libitum access to the autoclaved solid food CRF-1 (Oriental Yeast Co., Ltd.) and UV-irradiated tap water.

IV. Identification of Individual Animals and Cages

Animals were identified by individual numbers marked on the following day of arrival on the dorsal tail head using an oil-based marker. Cages were identified by labels with individual numbers from arrival to allocation, and by color labels carrying the study number, administration type, dose level and animal number after allocation.

V. Animal Allocation

For each study, the mean systolic pressure was calculated from the (systolic) blood pressure value measured during the acclimatization period. After excluding four animals each deviating from the mean blood pressure value, the rats were divided into the groups below, such that the blood pressure and body weight group means would be the same as much as possible. Rats in each group were marked with animal numbers. Extra animals were excluded from the study system after allocation.

VI. Administration

(i) Study Groups, Dose Levels and Dosing Periods

The rats were allocated to the four study groups (each of ten animals, n=10) below.
(1) Group receiving water for injection JP (control group)
(2) Group receiving CM6271 (300 mg/kg)
(3) Group receiving hydralazine (5 mg/kg) and water for injection JP (hydralazine group)
(4) Group receiving hydralazine (5 mg/kg) and CM6271 (300 mg/kg) (CM6271+hydralazine group)

CM6271 and hydralazine were administered orally everyday for two consecutive weeks from 10 weeks of age when the increase in blood pressure becomes mild.

(ii) Administration Methods

Study materials were given to rats once daily (in the morning) for two consecutive weeks by forced oral administration using a disposable syringe and gastric probe.

The dose volume was 5 mL/kg for CM6271 and water for injection JP, and 1 nL/kg for hydralazine, and the dose was based on the most recent body weight data. The same dose volume of the vehicle, or water for injection JP, was given to the control group.

VII. Experiments and Evaluation

The following experiments and evaluations were performed using the four rat groups (each of ten animals, n=10) described above.

For statistical analysis of blood pressure, pulse rate and body weight data, Dunnett test was used to make intergroup comparisons, and the level of significance was less than 5% (p<0.05) and less than 1% (p<0.01).

[General Observations, Body Weight Measurements, Food Intake]

The animals were observed for their general state once daily at dosing and their body weights were measured once a week, including the first and last days of dosing.

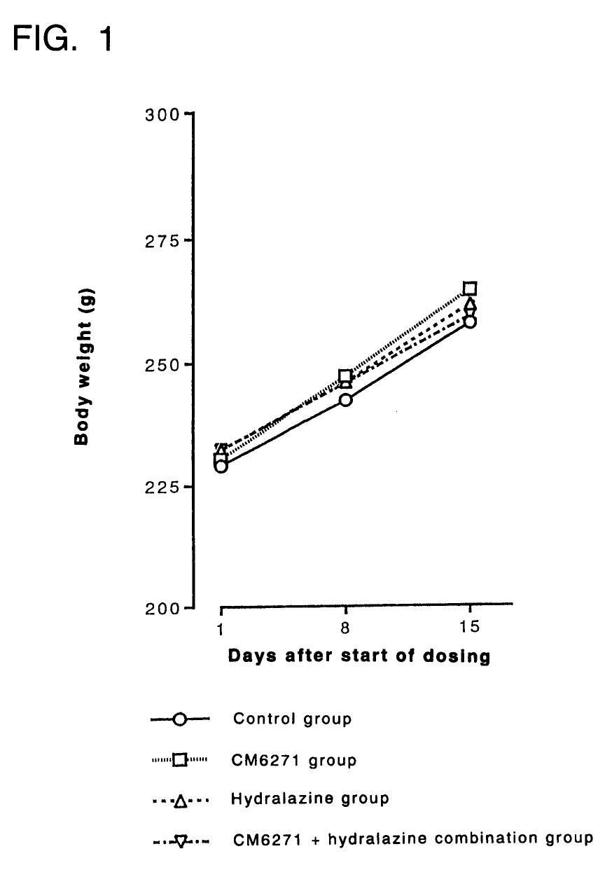

Body weight data are shown in Table 1 and FIG. 1. Figures in Table 1 represent mean±SD (standard deviation).

TABLE 1

| | Body weight (g) | | | |
|---|---|---|---|---|
| Group | Day 1 | Day 8 | Day 15 | Day 22 |
| Control group | 229.1 ± 17.5 | 242.3 ± 18.0 | 257.8 ± 18.3 | 274.1 ± 20.7 |
| CM6271 group | 230.4 ± 11.9 | 247.2 ± 13.9 | 264.5 ± 13.5 | 279.0 ± 13.6 |
| Hydralazine | 232.3 ± 16.1 | 246.0 ± 12.5 | 261.5 ± 13.7 | 276.1 ± 12.8 |
| group | | | | |
| CM6271 + | 232.3 ± 8.6 | 245.8 ± 9.1 | 259.2 ± 8.6 | 273.2 ± 9.0 |
| hydralazine | | | | |
| group | | | | |

As shown in Table 1 and FIG. 1, no significant differences were observed in bodyweight changes between the control and dose groups.

As to general observations, one case of the CM6271+hydralazine group showed nail peeling and reddening of the nail peeling site. Soft stools were observed in some animals of the control and hydralazine groups. No other changes were observed.

[Blood Pressure and Pulse Rate]

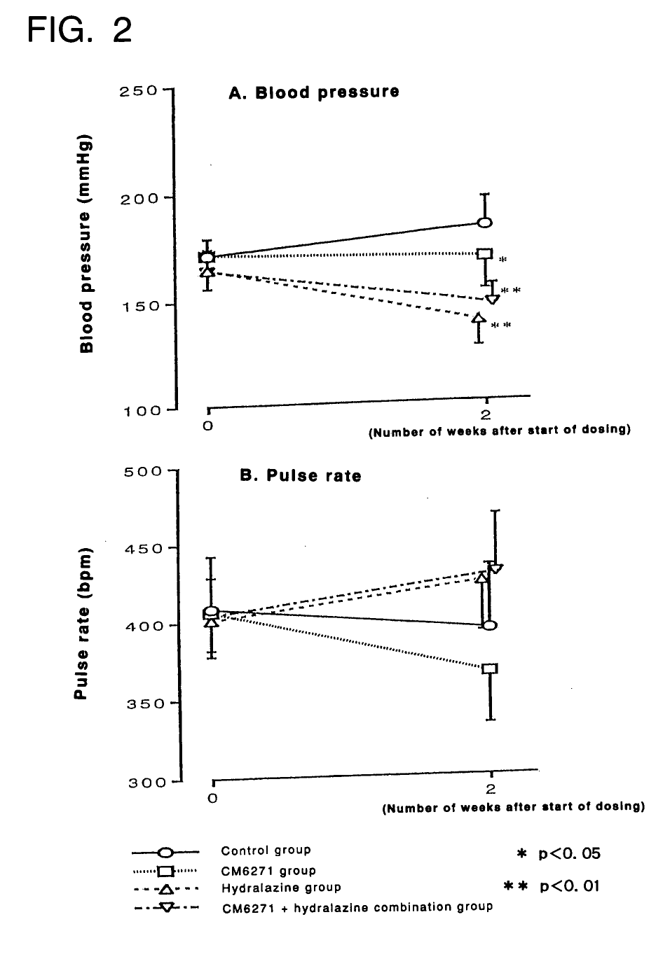

Systolic blood pressure and pulse rate were measured in all animals by the tail cuff method using an automated noninvasive sphygmomanometer (BP-98A, Softron Co., Ltd.) once during the acclimatization period and 23 to 25 hours after dosing in the second dosing week. Results are shown in Table 2 and FIG. 2. Figures in Table 2 represent mean±SD. In FIG. 2, ◯ mark represents the control group, the □ mark represents the CM6271 group, the Δ mark represents the hydralazine group, and the ∇ mark represents the CM6271+hydralazine group.

TABLE 2

| | Blood pressure (mmHg) | | Pulse rate (bpm) | |
|---|---|---|---|---|
| | Before | 2nd dosing | Before | 2nd dosing |
| Group | dosing | week | dosing | week |
| Control group | 171.6 ± 7.8 | 182.8 ± 13.0 | 408.6 ± 20.0 | 393.1 ± 41.0 |
| CM6271 group | 171.7 ± 8.7 | 168.4 ± 15.0* | 406.3 ± 28.5 | 365.1 ± 32.8 |
| Hydralazine group | 164.9 ± 8.8 | 138.4 ± 12.1** | 401.3 ± 19.4 | 423.6 ± 31.9 |
| CM6271 + hydralazine | 164.3 ± 10.1 | 146.4 ± 9.2** | 404.4 ± 38.1 | 428.7 ± 38.0 |
| group | | | | |

*p < 0.05 and
**p < 0.01 (vs. control group)

As shown in Table 2 and FIG. 2, mean systolic blood pressure increased from 171.6 mmHg before dosing to 182.8 mmHg after two weeks of dosing in the control group while it decreased from 171.7 mmHg to 168.4 mmHg in the CM6271 group with a significant difference from the control group.

In the hydralazine group, the blood pressure clearly decreased from 164.9 mmHg before dosing to 138.4 mmHg after two weeks of dosing with a significant difference from the control group. In the CM6271+hydralazine group as well, the blood pressure decreased from 164.3 mmHg to 146.4 mmHg with a significant difference from the control group, although no significant difference from the hydralazine group was observed.

In the control group, the pulse rates before dosing and after two weeks of dosing were almost the same; the pulse rate was 408.6 bpm before dosing and 393.1 bpm after two weeks of dosing. In the CM6271 group, the pulse rate decreased slightly from 406.3 bpm before dosing to 365.1 bpm after two weeks of dosing although the difference was insignificant.

In the hydralazine group, on the other hand, the pulse rate slightly increased from 401.3 bpm before dosing to 423.6 bpm after two weeks of dosing, and a similar tendency was observed in the CM6271+hydralazine group with 404.4 bpm before dosing and 428.7 bpm after two weeks of dosing.

### VIII. SUMMARY

An increase in mean systolic blood pressure was observed in the control group while the increase was inhibited in the CM6271 group, and a significant difference was observed after two weeks of dosing. The pulse rate was also slightly lower in the CM6271 group than in the control group.

In the hydralazine group, a significant decrease in blood pressure was observed after dosing started compared to the control group, and a similar-level decrease in blood pressure was also observed in the CM6271+hydralazine group. No difference in pulse rate was observed either between the hydralazine group and the CM6271+hydralazine group.

It was concluded that the oral administration of CM6271 at 300 mg/kg showed a hypotensive action on SHR rats, which develop hypertension spontaneously, and in combination with hydralazine at 5 mg/kg, did not affect the hypotensive action of hydralazine.

1. A hypotensive agent containing Tricholoma matsutake or an extract thereof.

2. The hypotensive agent according to claim 1, wherein said T. matsutake is provided in the form of mycelia, broth or fruit bodies including spores.

3. The hypotensive agent according to claim 1, wherein said T. matsutake is strain FERM BP-7304.

4. The hypotensive agent according to claim 1, wherein said T. matsutake is a dried mycelial powder of strain FERM BP-7304.

5. The hypotensive agent according to claim 1, wherein said T. matsutake extract is hot water or aq. alkali extract from mycelia of the strain FERM BP-7304.

6. A method of treating hypertension which comprises administrating to a human or an animal in an effective amount of the hypotensive agent of any one of claims 1-5.

7. A hypotensive food containing Tricholoma matsutake or an extract thereof.

8. The hypotensive food according to claim 7, wherein said T. matsutake is provided in the form of mycelia, broth or fruit bodies including spores.

9. The hypotensive food according to claim 7, wherein said T. matsutake is strain FERM BP-7304.

10. The hypotensive food according to claim 7, wherein said T. matsutake is a dried mycelial powder of strain FERM BP-7304.

11. The hypotensive food according to claim 7, wherein said T. matsutake extract is hot water or aq. alkali extract from mycelia of the strain FERM BP-7304.

12. A method of treating hypertension which comprises the intake of by a human or an animal in an effective amount of the hypotensive food of any one of claims 7-11.
